(19) United States
(12) Patent Application Publication
(10) Pub. No.: US 2014/0303453 A1
(43) Pub. Date: Oct. 9, 2014

(54) System and Method for Generating Composite Measures of Variability

(71) Applicants: Andrew E. Seely, Ottawa (CA); Andrea Bravi, Ottawa (CA); André Longtin, Ottawa (CA)

(72) Inventors: Andrew E. Seely, Ottawa (CA); Andrea Bravi, Ottawa (CA); André Longtin, Ottawa (CA)

(73) Assignee: ONTARIO HOSPITAL RESEARCH INSTITUTE, Ottawa, ON (CA)

(21) Appl. No.: 14/131,081

(22) PCT Filed: Jun. 28, 2012
(86) PCT No.: PCT/CA2012/050440

§ 371 Date: Jan. 6, 2014

Related U.S. Application Data
(60) Provisional application No. 61/504,791, filed on Jul. 6, 2011.

(51) Int. Cl. A61B 5/00

(52) U.S. Cl. CPC A61B 5/7282; A61B 5/7242
USPC 600/301

(57) ABSTRACT

A system and method are provided for assessing the degree of critical illness of patients in Intensive Care Units (ICUs), and the detection of occurrence of specific clinical outcomes, pursued through the monitoring of a variety of physiological signals, extraction of multiple measures of variability, and integration of the relevant information the measures of variability bring. The integration process leads to the creation of a compound variable, referred to as a composite measure of variability, which can be tuned to various clinical needs, since the process of normalization, feature selection and data integration are customizable to the clinical indication, planned use of the composite variability monitoring. The system and method also enable joining the composite measure of variability with clinical factors, so as to estimate the likelihood of developing specific clinical outcomes.

This application claims priority to U.S. Provisional Patent Application No. 61/504,791, filed Jul. 6, 2011, the contents of which are incorporated herein by reference.

### TECHNICAL FIELD

The following relates generally to generating composite measures of variability, and more particularly to generating composite measures of variability for clinical applications.

### BACKGROUND

There is an increasing interest in the application of variability monitoring to improve clinical outcomes. For example, there has been extensive research into the development of multiple measures of variability and the characterization of how variability changes with respect to specific stimuli. However, several steps need to be made to create a monitoring system capable of tracking the physiological conditions of patients undergoing different types of stresses, either physiological (e.g. physical exercise) or pathological. In particular, the following challenges need to be addressed.

The first challenge is in the characterization of the nature and degree of interdependence between measures of variability in specific clinical situations. The common approach in the literature is reductionist, wherein just a few variability measures, often of the linear type, are selected among the huge array that the literature currently offers. Heart rate variability is a good example of a phenomenon that has been studied with a few nonlinear techniques at a time, such as the Recurrence Plot and Poincare plot, which reveal phase space structure; Point-process techniques, which focus on the stochastic nature of the underlying process; and Multi-Scale Entropy, which investigates regularities across different time scales. However, it is a well-accepted idea that there are many domains of variability (i.e. time, frequency, scale-invariant, etc.) holding clinically valuable information, and that a better assessment of clinical situations requires joining the many pieces of information that each single technique can provide. A broad comparison of the variability techniques accessible today in specific clinical settings would enable an understanding of the independent value of each and of their overlap. It would also simplify the decision of choosing a particular set of techniques, thereby providing the basis for an adaptable system for variability monitoring.

The second challenge is in the extension of variability analyses to multivariate techniques characterizing multi-organ variability. The common approach is to use linear uni-variate techniques, often combined with a small set of nonlinear uni-variate techniques. Examples of multivariate studies exist, and are mainly focused on the evaluation of interactions between the cardiac and respiratory systems through frequency analysis (spectral, wavelet . . . ), or the evaluation of the differences between heart rate variability and blood pressure, or pulse oximeter data, which measures the oxygen saturation in the blood. A challenge that variability research needs to face is to consider the different components of a physiological system not independently, but as a whole. Several multivariate techniques are accessible but their application remains rare in clinical settings. Examples are the multivariate state space representation, Cross Recurrence Plots, Cross Fuzzy Entropy, and Multidimensional Modeling.

It is an object of the following to address the above-noted challenges.

### SUMMARY

In one aspect, there is provided a method comprising: obtaining a plurality of measures of variability; and combining the plurality of measures of variability to obtain a composite measure of variability.

In another aspect, there may be provided a computer readable medium comprising computer executable instructions for performing the above method.

In yet another aspect, there is provided system comprising a processor coupled to a memory, the method comprising computer executable instructions for performing the above method.

### BRIEF DESCRIPTION OF THE DRAWINGS

Embodiments will now be described by way of example only with reference to the appended drawings wherein:

FIG. 1 is a block diagram of a system for generating composite variability measures.

FIG. 2 is a block diagram of a hospital site including a variability analysis server.

FIG. 3 is a block diagram of a clinic site including a variability analysis server.

FIG. 4 is a block diagram of a mobile site including a variability analysis server.

FIG. 5 is a block diagram illustrating an example of a process flow for generating a composite variability output.

FIG. 6 illustrates a set of variability time series graphs.

FIG. 7 illustrates a set of filtered variability time series graphs.

FIG. 8 illustrates a set of normalized variability graphs.

FIG. 9 illustrates an integration of the normalized variability graphs of FIG. 8.

FIGS. 10 and 11 are graphs illustrating a population-based normalization.

FIG. 12 is a flow chart illustrating an example of a set of computer executable operations that may be performed in using a composite variability output to determine a clinical likelihood.

FIG. 13 illustrates a generic logistic regression model.

FIG. 14 is a chart illustrating an example of a Principal Component Analysis (PCA) composite variability trend output tracking the development of sepsis wherein time t=0 corresponds to the diagnosis of sepsis.

FIG. 15 is a chart illustrating an example of a median composite variability trend output during the monitoring of a patient who failed extubation, pre- and during a spontaneous breath trial wherein time t=0 corresponds to the beginning of the spontaneous breath trial.

FIG. 16 is a chart illustrating an example of a median composite variability trend output during the monitoring of a patient who succeeded extubation, pre- and during a spontaneous breath trial wherein time t=0 corresponds to the beginning of the spontaneous breath trial.

FIG. 17 is an example of a population trend for a Shannon Entropy (shannEn) measure of variability.

FIG. 18 is an example of a population trend for a Plotkin-Swamy engery operator (PSeo) measure of variability.

FIG. 19 is an example of a population trend for a Fuzzy Entropy (fuzEn) measure of variability.

### DETAILED DESCRIPTION OF THE DRAWINGS

It will be appreciated that for simplicity and clarity of illustration, where considered appropriate, reference numerals may be repeated among the figures to indicate corresponding or analogous elements. In addition, numerous specific details are set forth in order to provide a thorough understanding of the example embodiments described herein. However, it will be understood by those of ordinary skill in the art that the example embodiments described herein may be practised without these specific details. In other instances, well-known methods, procedures and components have not been described in detail so as not to obscure the example embodiments described herein. Also, the description is not to be considered as limiting the scope of the example embodiments described herein.

### Composite Measures of Variability for Clinical Applications

In addition to the above described challenges, it has been recognized that a third challenge needs to be addressed in tracking the physiological conditions of patients undergoing different types of stresses. The third challenge is in the creation of a composite measure of variability to detect critical changes in health status. The collection of several independent measures of organ and multi-organ variability calls for easy-to-use metrics describing the physiological condition of a patient. Considering clinical settings, the application of this type of analysis represents an old problem in fields like EEG analysis, but a new one in the case of multi-organ variability analysis.

The following relates to the assessment of the degree of critical illness of patients in Intensive Care Units (ICUs), and the detection of occurrence of specific clinical outcomes, pursued through the monitoring of a variety of physiological signals, extraction of multiple measures of variability, and integration of the relevant information the measures of variability bring. As will be discussed below, the integration process leads to the creation of a compound variable, which can be tuned to various clinical needs, since the process of normalization, feature selection and data integration are customizable to the clinical indication, planned use of the composite variability monitoring. The compound variable may be hereinafter referred to as a composite measure of variability. The following also presents how to join the composite measure of variability with clinical factors, so as to estimate the likelihood of developing specific clinical outcomes.

An object of the following is therefore to provide a procedure unifying the clinically relevant information from multiple measures of variability.

It can be appreciated that the measures of variability that are applicable to the following techniques include both single-organ and multi-organ variability measures. Furthermore, the following provides a procedure taking into account the information extracted from a single physiological signal, as well as a variety of physiological signals. These include, without limitation: electrocardiogram, end tidal capnography, oxygen saturation and arterial blood pressure waveforms, and their derived time series (e.g. R—R interval, inter-breath interval, pulse transit time). As such, the following joins clinically relevant information from multiple variability signals extracted from a variety of physiological signals, creating a composite measure of variability.

The composite measure of variability may be created by applying the following procedures: filtering, normalization, selection of relevant information, and integration. The principles described herein provide for the development of a composite measure of variability which is tunable depending on the clinical needs. In particular, the types of normalization, selection, and integration can be chosen according to a purpose of use.

The proposed filtering methods may include, without limitation, Savitzky-Golay filter, Butterworth filter, Bessel filter, elliptic filter and Chebyshev filter, etc.

The proposed normalizations may include, without limitation, normalization with respect to the admission conditions, population-based normalization, and [−1,+1] normalization.

The proposed selection criteria may include, without limitation: domain-specific selection, physiologically-based selection, and clinically relevant selection.

The proposed integration techniques may include, without limitation: median, principal component analysis, independent component analysis, k-means clustering, fuzzy c-means clustering, artificial neural networks, multilayer perceptron, radial basis function network, support vector machines, decision trees, random forests, generalized linear models, and partial least square regression.

As such, the following system and method are capable of tracking the conditions of the patient, providing an individualized monitoring tool which makes use of population information, and can be tuned according to specific clinical needs.

The following also provides the capability of estimating, from the composite variability, the likelihood of developing a specific clinical outcome (i.e. a clinical likelihood). Such a clinical likelihood can be estimated by joining the information of the composite variability with the information from clinical factors (e.g. body temperature, Body Mass Index, etc.), and can be used to describe the conditions of the patient at the moment of investigation. The technique used to estimate the clinical likelihood may be, for example, logistic regression.

The clinical outcomes that are addressed by the composite measures that may be generated according to the principles below include, without limitation: extubation outcome from mechanically assisted breathing, spontaneous breath trial outcome, development and degree of sepsis, the onset of septic shock, development and degree of multiple organ dysfunction syndrome, and the degree of physical exercise. As such, the following provides different types of composite measures of variability; those measures are tunable to the specific clinical needs of a patient, and can estimate the likelihood of developing specific clinical outcomes. Moreover, the composite variability and the clinical likelihood represent two modules, which can be provided alone or together with others inside a monitoring system.

Accordingly, the following principles enable: the combining of single organ and multiple organ variability measures into one composite measure; the tracking of a composite variability measure over time; the combining of variability measures into one composite measure reflecting the overall degree of change in the physiological conditions; the combining of variability measures into one composite measure correlating with specific clinical outcomes; the combining of variability measures into one composite measure sensitive to the physiological and pathological stimuli causing variability; the combining of composite measures of variability with clinical information to produce likelihood of development of specific clinical outcomes; the tuning of composite measures of variability to detect specific clinical outcomes, through the selection of a variety of normalization, selection and integration procedures applied to variability measures; the tuning of composite measures of variability to investigate domains of variability, through the use of a variety of normalization, selection and integration procedures applied to variability measures; and the tuning of composite measures of variability to investigate the physiological phenomena underlying variability, through the use of a variety of normalization, selection and integration procedures applied to variability measures.

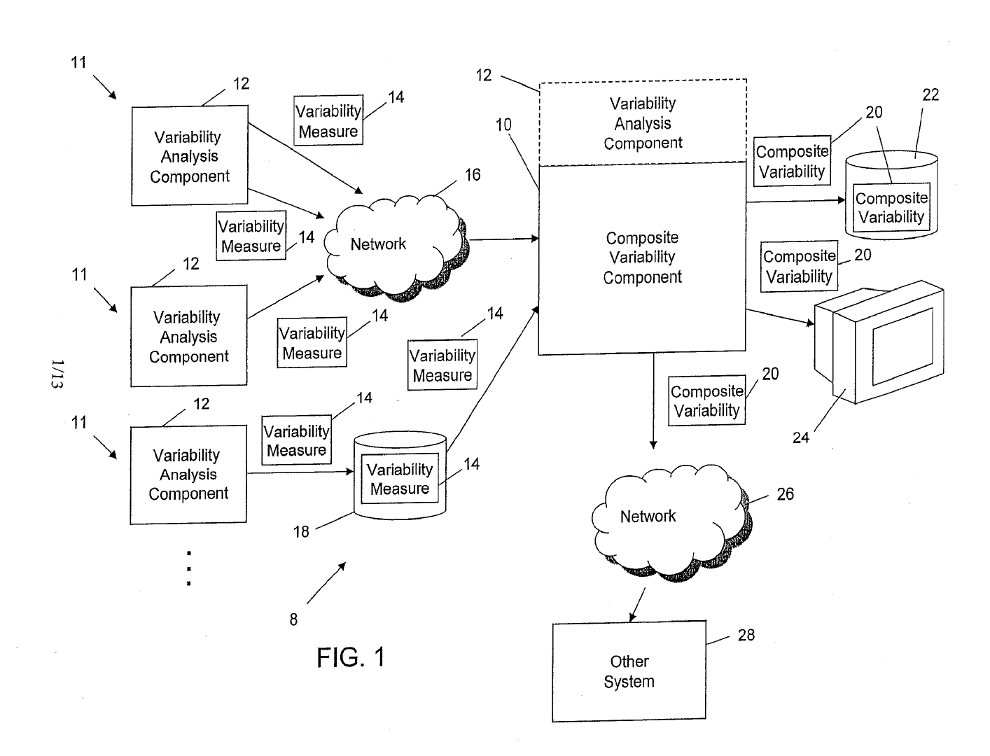

Turning now to the figures, FIG. 1 illustrates an exemplary system 8 for obtaining multiple measures of variability (also variability measure) 14, and generating a composite measure of variability (also composite variability) 20 therefrom. In the example embodiment shown in FIG. 1, several variability analysis components 12 are shown, each at a corresponding monitoring site 11. Each variability analysis component 12 comprises software, hardware, or a combination thereof for monitoring variability of physiological parameters associated with one or more organ systems and computing or otherwise generating variability measures 14. FIG. 1 illustrates that the variability measures 14 may be generated and sent over a network 16, stored in a data store 18 for later use, and multiple variability measures may be obtained from the same variability analysis component 12 (see uppermost variability analysis component 12). It can be appreciated that the variability analysis component 12 may in general represent any server, computing system, clinical equipment, service, or other data component that is operable to execute computer executable instructions for generating the variability measures 14 using monitored physiological parameters. Further detail regarding how the variability measures 14 are computed and in which environments is provided in the next section.

The variability measures 14, whether sent over a network 16, or stored in a data store 18 and transported using another medium; are provided in this example embodiment to a composite variability component 10. It can be appreciated that as shown in FIG. 1, the composite variability component 10 may be coupled to, integrated with, or otherwise operable with a variability analysis component 12 and thus the separation between such components 10, 12 is shown for illustrative purposes only. The composite variability component 10 comprises software, hardware, or a combination thereof which is programmed or configured to utilize variability measures 14 to generate a composite variability 20. It can be appreciated that, similar to the variability analysis component 12, the composite variability component 10 may in general represent any server, computing system, clinical equipment, service, or other data component that is operable to execute computer executable instructions for generating a composite variability 20 using multiple variability measures 14.

As shown in FIG. 1, the composite variability 20 may be stored in a data store 22, provided to a monitor 24 for display purposes, or provided to another system 28 over a network, to name a few.

The following section provides further detail regarding various examples of how the variability measures 14 may be obtained.

### Obtaining Variability Measures

As discussed above, the composite measures of variability may be considered an extension of individual variability measures. As such, the generation of a composite measure of variability can be applied in any context in which variability measures, obtained from a variability analysis component 12, can be applied. For example, composite variability measures can be generated using data obtained in real-time, older data, data obtained in an intensive care unit (ICU), data obtained using portable monitoring devices recording variability, etc. A composite measure of variability therefore is not dependent on any particular mechanism for obtaining the variability data, so long as a set of variability measures is available, and a composite measure can be obtained, as explained in greater detail below. The following illustrates three exemplary monitoring sites 11 to demonstrate the various ways in which the variability measures 14 can be obtained in order to generate a composite variability measure 20. Further detail concerning an underlying software framework for obtaining and distributing variability data can be found in applicant's co-pending U.S. patent application Ser. No. 12/752,902, published under US 2010/0261977 to Seely, the entire contents of which are incorporated herein by reference.

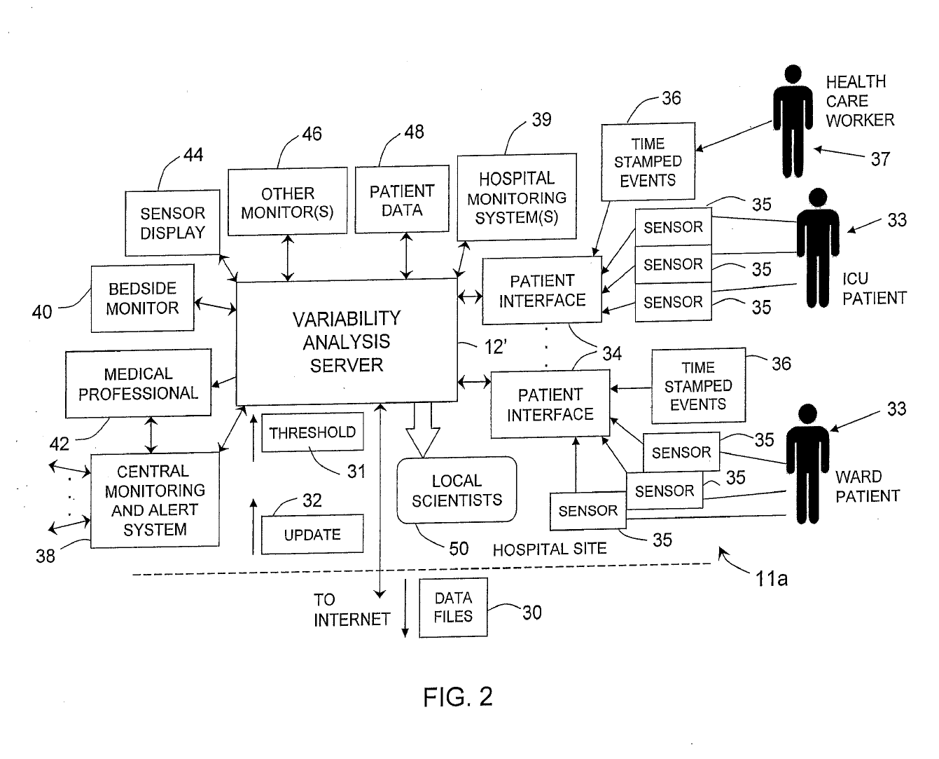

An example of a hospital monitoring site 11a is shown in FIG. 2. The elements shown in FIG. 2 are meant to illustrate several possible components that may interact with one another at the hospital site 11a, however, any number (or all) of these elements can be used or not used in specific hospital sites 11a depending on the actual equipment and/or personnel present at the hospital site 11a and the needs of the patients 33 and personnel. In addition, the parameters being monitored (and the monitors themselves) may differ from network to network. As will be explained, at each monitoring site 11, including the hospital site 11a shown in FIG. 2, is at least one variability analysis server 12′ for using acquired data to conduct variability analyses over time and generate data files 30 that can be viewed at the site and provided to, for example, a central service (not shown). However, as shown, each variability analysis server 12′ can interface with multiple patients 33 and, as such, typically only one variability analysis server 12′ is required at each monitoring site 11. The variability analysis server 12′ gathers data acquired from one or more patients 33 through individual patient interfaces 34, computes the measures of variability (i.e. conducts variability analyses) for one or more patient parameters, and connects to, for example, a central server through the Internet, for facilitating the transfer and/or receipt of data files 30, threshold data 31 and update data 32. As shown, there can be different types of patients 33 such as those in the ICU or in a regular hospital ward.

The patient interfaces 34 monitor physiological parameters of the patient 33 using one or more sensors 35. The data or patient parameters can include any variable that can be accurately measured in real time or intermittently. The data may be obtained from a continuous waveform (at a certain frequency level, e.g. 100 Hz for a CO2 capnograph or 500 Hz for an EKG), or taken as absolute measurements at certain intervals, e.g. temperature measurements. The sensors 35 and patient interfaces 34 may include, for example, an electrocardiogram (ECG), a CO₂ capnograph, a temperature sensor, a proportional assist ventilator, an optoelectronic plethymography, a urometer, a pulmonary arterial catheter, an arterial line, an O₂ saturation device and others. To provide more meaning to the data acquired through the sensors 35, clinical events are associated with the data, through an act of recording time stamped events 36, which are typically entered by a heath care worker 37 in the hospital (bedside) environment. Clinical (time stamped) events can be physical activity, administration of medication, diagnoses, life support, washing, rolling over, blood aspiration etc. The clinical events are associated with a specific time, which is then also associated with the data that is acquired at the same specific time using the sensors 35. It will be appreciated that the clinical events can also be recorded in an automated fashion, e.g. by utilizing algorithms which detect events electronically and process such events to designate them as clinical events or noise. In this example, the patient interface 34 is configured to gather the time stamped event data 36 concurrently with the sensor data 35, further detail being provided below. It may be noted that additional non-time-stamped information (e.g. demographics) can also be recorded for each patient.

As can be seen in FIG. 2, the variability analysis server 12′ not only connects to the patient interfaces 34 and the Internet, but also to several other components/entities within the hospital site 11a. For example, the server 12′ can interface with a hospital monitoring system 39 such as a nurse's station, as well as a central monitoring and alert system 38. The central monitoring and alert system 38 is capable of monitoring the variability analyses performed by the variability analysis server 12′ in order to detect critical or potentially critical situations evident from such variability analyses and provide an alert or alerts to a medical professional 42, who can also receive data directly from the variability analysis server 12′. The variability analysis server 12′ can be embodied as a fixed unit or a moveable unit such as on a cart, in order to facilitate movement about the hospital site 11a to serve multiple patients 33 in multiple locations. Similarly, the variability analysis server 12′ can be a proprietary apparatus or can be embodied as an add-on to existing beside or centralized equipment to minimize space.

The variability analysis server 12′ can also interact with a bedside monitor 40, which may be made available to or otherwise represent a nurse or other personnel that monitors the patient 33 at the bedside. Similarly, the variability analysis server 12′ can also interact with sensor displays 44, which are associated with other medical equipment such as ECGs, blood pressure sensors, temperature sensors etc. As noted above, the variability analysis server 12′ can be a separate, stand-alone unit but may also be integrated as a plug-in or additional module that in this case could be used or integrated with existing bedside monitoring equipment, displays and sensors. FIG. 2 also shows other monitors 46 which can include any other monitoring system or equipment that either can provide useful medical data or patient data 48 or would benefit from the data acquired by the variability analysis server 12′. Patient data 48, e.g. provided by an electronic patient database (not shown) or manually entered can also interact with the variability analysis server 12′. As will be discussed below, the patient data 48 may be appended to, or included with the data files 30 to provide further context for the data contained therein. This enables patient specifics such as age, general health, sex etc. be linked to the acquired data to assist in organizing data into demographics. As also shown in FIG. 2, the variability analysis server 12′ can provide data or otherwise useful information for local scientists 50 that are interested in or involved in the implications and effects of variability. It will be appreciated that patient privacy and other concerns can be addressed as required, by adding data security or other de-identification measures.

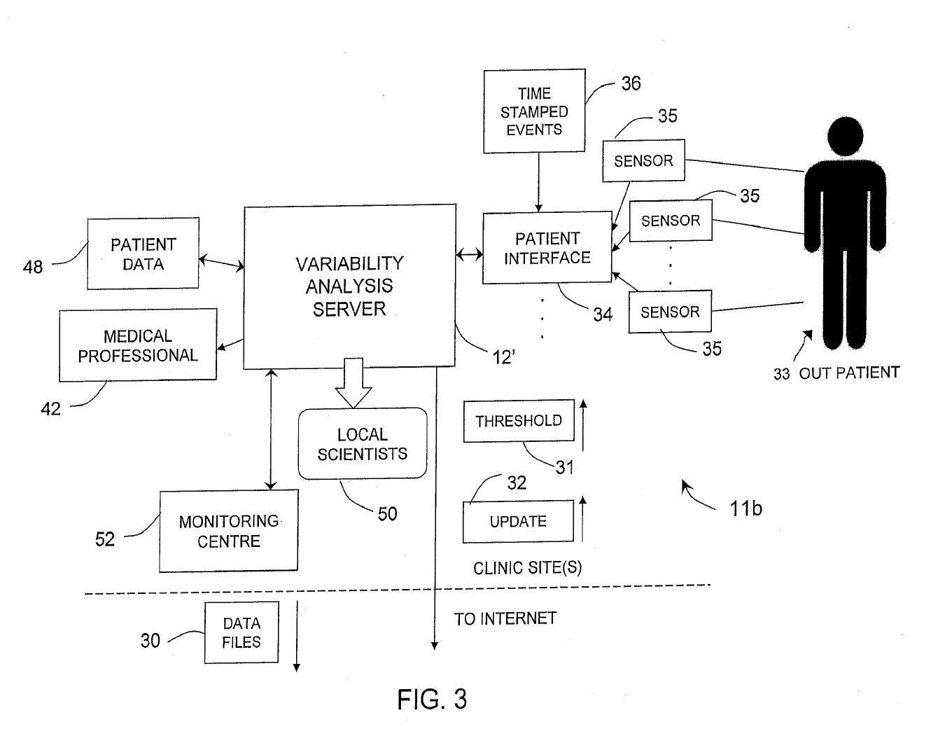

Turning now to FIG. 3, a clinic site 11b is shown. An example of a clinic site 11b is a bone marrow transplant clinic. Similar to the hospital site 11a discussed above, the clinic site 11b includes a variability analysis server 12′, that obtains data from one or more patient interfaces 34, and connects to the Internet for facilitating data transfer (i.e. to send data files 30 and to receive threshold data 31 and update data 32). In the clinic site 11b, the patients 33 are referred to as outpatients as they are not admitted to a hospital. The sensors 35, clinical events recorded as time stamped events 36 and patient data 48 is acquired and used in a manner similar to that discussed above and thus further details need not be reiterated. Similarly, the variability analysis server 12′ can provide data and interact with medical professionals 42 at the clinic site 11b, as well as local scientists 50, if applicable. The clinic site 11b may include one or more variability analysis servers 12′, and would typically include a monitoring centre 52 that monitors the analyses of the various outpatients 33 and provides alerts if necessary. The monitoring centre 52 enables the clinic's variability analysis server 12′ to be monitored from a remote location and allows personnel to monitor several servers 12′ if several are present in the clinic. In this way, a central monitoring centre 52 can be used to service several clinic sites 11b.

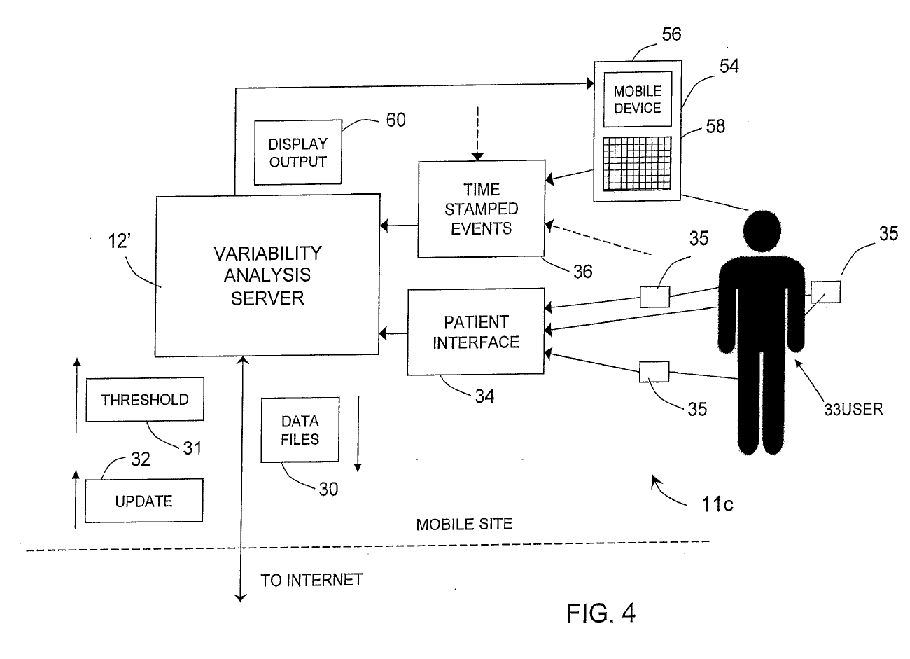

A mobile site 11c is shown in FIG. 4. The mobile site 11c enables the capabilities of the variability analysis server 12′ to be used outside of the hospital and clinical environments and, as such, in this embodiment, the mobile site 11c serves any “user” or “subject”. For the sake of consistency, hereinafter the term “patient” will refer collectively to any user or subject. In this way, it may be appreciated that variability analyses can be performed on any user, including athletes, firefighters, police officers, or any other person that can benefit from monitoring variability of one or more physiological parameters. This can therefore extend to providing real-time monitoring in extreme environments such as during a fire, in a mine, during rescue missions etc. where variability can indicate a potentially critical situation. In all cases, variability can be monitored over time and analyzed on an individual basis for any patient 33 such that the resultant data is specific to that individual. Using the wider system 8 allows a central service to take advantage of the individual results for many patients 33 and ascertain further and more complete information. The mobile site 11c generally represents any site that includes a variability analysis server 12′, which connects to the system 8 and can communicate with one or more patients 33, whether they are patients in the traditional sense or another type of user.

In the example shown in FIG. 4, the user 33 generally includes a mobile device 54 and has a number of sensors 35 that are in communication with a variability analysis server 12′. The mobile device 54 can also be used to provide inputs, e.g. for the time stamped event data 36, as well as to provide a display to the user 33 for entering parameters or to view display data 60 acquired by the sensors 35 and/or processed by the server 12′. The connections between the mobile device 54 and the server 12′, as well as between the sensors 35 and patient interface 34 can be wired or wireless and the variability analysis server 12′ can be a fixed unit at a base station or a portable unit such as on a cart at a monitoring centre. The mobile device 54 can be a personal digital assistant (PDA), mobile telephone, laptop computer, tablet computer, personal computer, or any other device that can provide an input device, a display and some form of connectivity for interacting with the variability analysis server 12′, preferably in a completely mobile manner.

As noted above, each monitoring site 11 may include a variability analysis server 12′. Details of various embodiments of existing variability analysis apparatus and configurations can be found in U.S. Reissue Pat. No. RE41,236 E to Seely, the entire contents of which are incorporated herein by reference.

### Generating Composite Variability Measures

The origin and nature of the variability measures 14 will depend on the clinical application. The following describes how multiple variability measures 14 may be integrated to generate a composite variability 20. A set of variability time series may be extracted through a windowed analysis of one or more physiological signals. For example, a moving window approach may be used, wherein variability measures 14 are obtained over a particular window (e.g., 5 minutes), and a step forward of a particular amount of time (e.g., 2.5 minutes) is applies and the process repeated.

The physiological signals can be extracted in real time from acquisition devices (e.g. electrocardiograph, Holter monitor) as well as taken from a database, and are submitted to the processing unit (e.g. variability analysis server 12′) computing the variability algorithms. The algorithm for the composite variability 20 may then be implemented from these variability measures 14.

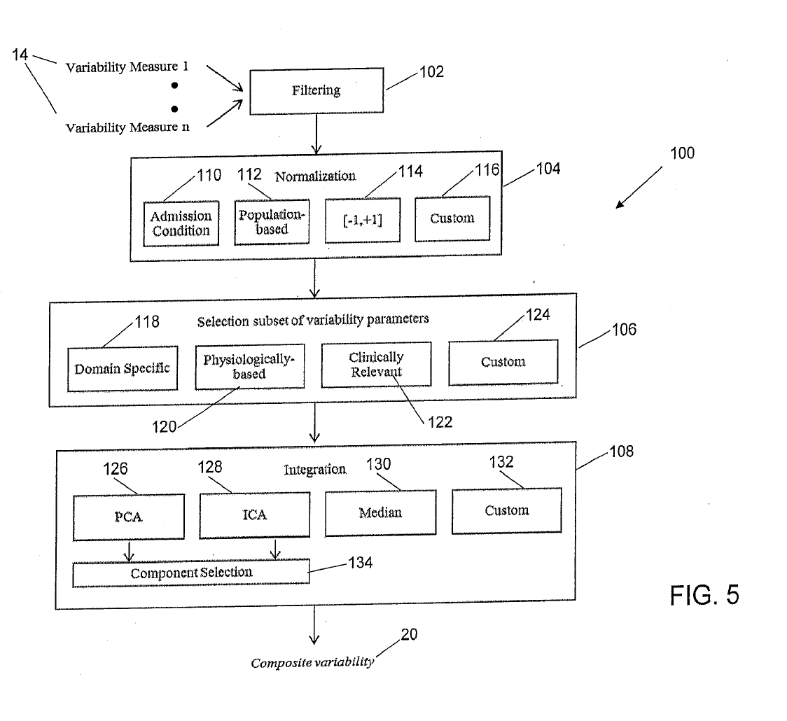

FIG. 5 illustrates a block diagram of an example of a composite variability process 100, which includes various signal processing operations on the variability time series to obtain the composite variability 20. As seen in FIG. 5, in this example embodiment, multiple variability measures 14 (e.g. variability measure 1 to variability measure N) are obtained and undergo a filtering stage 102. A normalization stage 104 is then applied to the filtered data, and a selection of a subset of variability parameters performed at stage 106. The outcome of the selection stage 106 then undergoes an integration stage 108 to arrive at the composite variability 20. Various examples of normalization processes are shown in FIG. 5, which include, without limitation, admission condition 110, population based 112, [−1,+1], and custom normalization 116. Various examples of selection processes that may be applied in stage 106, are shown in FIG. 5, which include, without limitation, a domain specific selection 118, a physiological-based selection 120, a clinically relevant selection 122, and a custom selection 124. Various examples of integration techniques are also shown in FIG. 5, which include, without limitation, a principal component analysis (PCA) technique 126, an independent component analysis (ICA) technique 128, a median technique 130, and a custom technique 132. Since PCA 126 and ICA 128 techniques create a number of time series equal to the number of time series provided to them, a component selection sub-stage 134 is also shown for enabling the selection of the component expressing the clinically relevant information. PCA 126 and ICA 128 are techniques allowing the representation of a dataset in another coordinate system. Therefore, the dimensionality of the problem remains unaltered (e.g. if PCA 126 is applied to 4 variability time series, 4 principal components are created). It can be appreciated that the different principal/independent components differ from each other, therefore the sub-stage 134 selection can be made according to for what the composite measure of variability is being used. For example, in sepsis detection it has been found that the first principal component is the most correlated with the development of sepsis, therefore that principal component is selected. It may be noted that the first component is oriented in the direction of maximum variation, which provides a good index of change. It can therefore be seen that in addition to supporting various processes and techniques, the stages performed by the composite variability component 10 can support custom techniques and processes for adapting to different applications.

It can be appreciated that the filtering and selection stages 102, 106 may be optional in some applications. Although the normalization stage 104 is also optional, the multiple measures may not be comparable, thus diminishing the meaningfulness of the output.

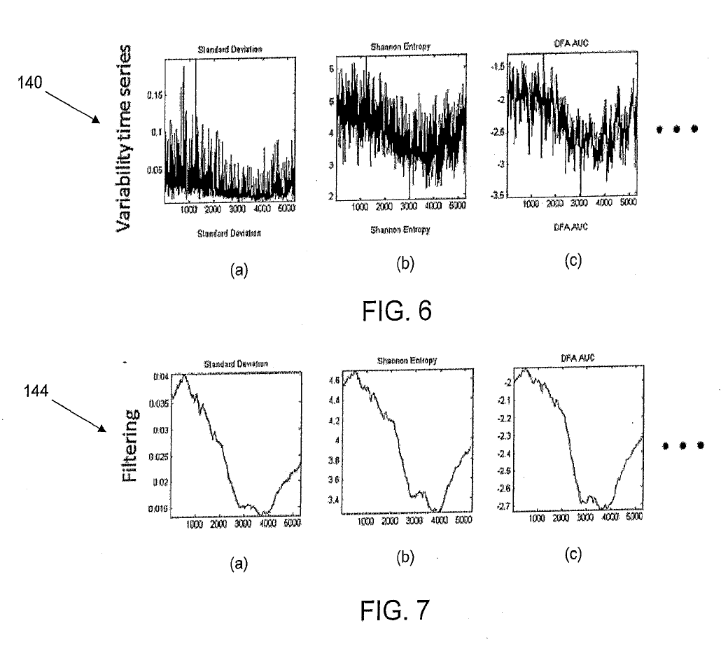

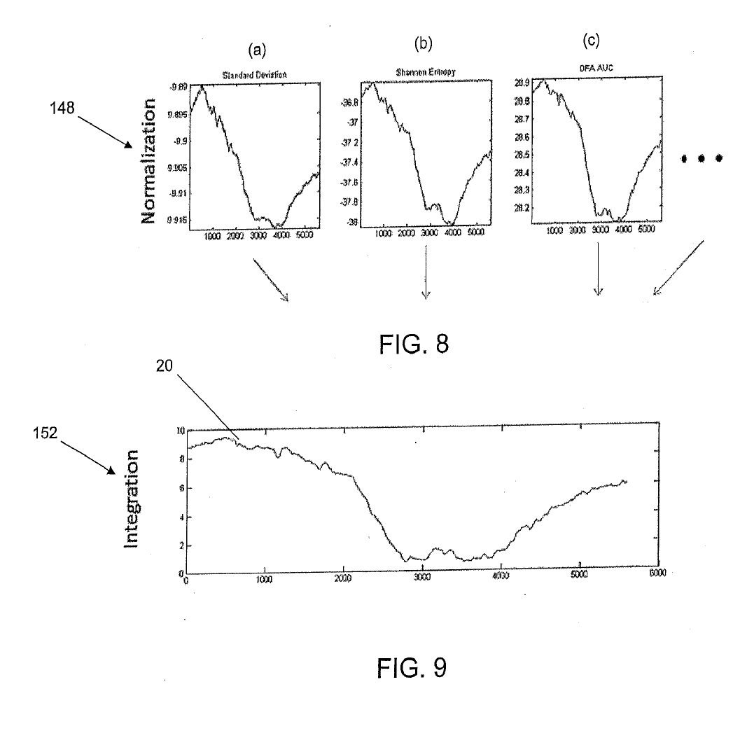

An example set of graphs illustrating the processing of variability time series 140 to obtain a composite variability 20 is shown in FIGS. 6 through 9. It can be appreciated that the composite variability processes described herein may include various ones of the operations shown in FIGS. 6 to 9, as well as other additional unspecified ones, since different applications may require different type of processing. In the example processing shown, FIG. 6 illustrates multiple variability time series 140 (a, b, c, etc.). Time series (a) has been acquired using a standard deviation variability technique, time series (b) has been acquired using a Shannon Entropy technique, and time series (c) has been acquired using a Detrendend Flucutation Analysis Area Under the Curve (DFA AUC) technique. The variability time series 140 after undergoing the filtering stage 102 result in multiple corresponding filtered outputs 144 (a, b, c, etc.) as shown in FIG. 7. The filtered outputs 144 are then subjected to the normalization stage 104 to obtain corresponding normalized outputs 148 (a, b, c, etc.) as shown in FIG. 8. FIG. 9 illustrates an integration 152 of the normalized outputs 148 thus providing a composite variability 20 in the graph shown in FIG. 9.

Each of the stages shown in FIG. 5 will now be described in greater detail.

In stage 102, each variability time series undergoes filtering through, e.g. a Savitzky-Golay first order filter to obtain the filtered outputs 144.

In the normalization stage 104, each filtered variability time series 144 undergoes a transformation that makes it comparable with the other filtered time series 144. Depending on the type of study, one or more of the following normalization techniques can be considered. In the following examples, consider a generic time series x(5), where n relates to the n-th sample of the time series.

1. Normalization with respect to admission conditions 110:

$$x ( n ) = \frac{x ( n ) - μ}{σ}$$

In this technique, is the sample mean, and the sample standard deviation of the time series during a specific time after admission (e.g. from admission to 6 hours later). This normalization creates a relative measure, i.e. a measure which emphasizes the changes from a zero (in this case the admission conditions). Therefore, it measures the deterioration from a baseline of health. A possible use is the detection of sepsis after transplant. It may be noted that it has been shown that patients after transplant start with healthy conditions (high variability), and deteriorate with time, causing a continuous drop in variability; this variability was shown to track the development of sepsis.

2. Population-based normalization 112

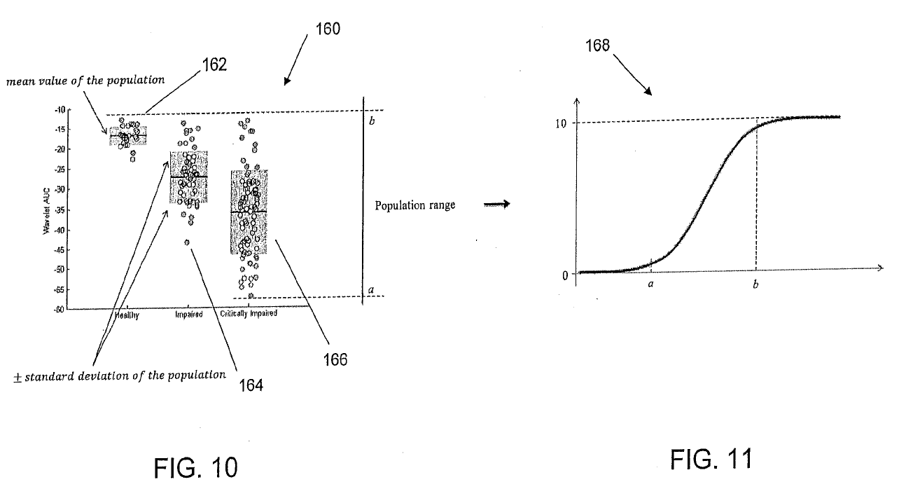

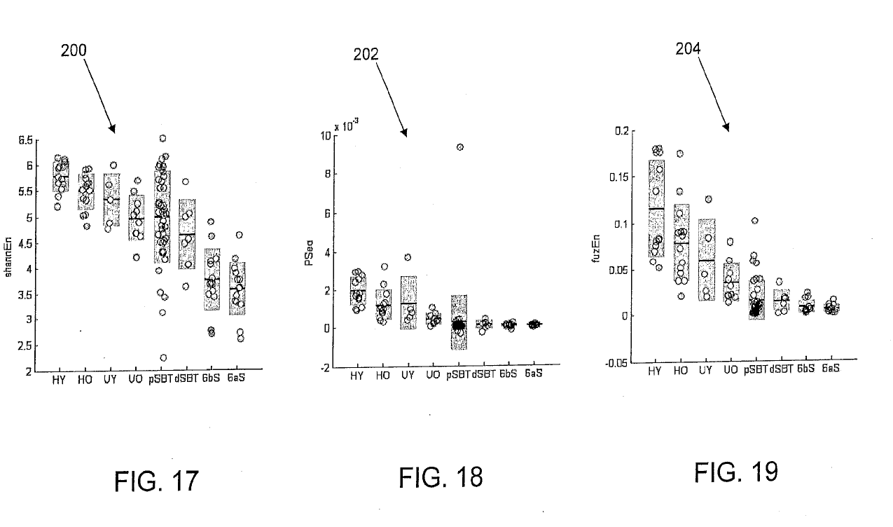

In this technique as shown in the graph 160 in FIG. 10, for each variability measure, the population cut-offs are taken from the characterization of the variability in healthy 162, impaired 164, and critically ill patients 166. Then, as shown in FIG. 11, the values are normalized in a scale from 0 to 10 using, for example, a sigmoid function. This normalization enables the direct association of variability to the degree of impairment, creating an absolute measure. Therefore, the emphasis is not on the changes from a starting point (zero) as in the first technique, but rather on the severity of illness. It may be noted that this normalization technique is possible because there exists a monotone relationship between certain measures of variability and the degree of impairment (three examples are provided in FIGS. 17 through 19).

3. Normalization between [−1,+1] 114, according to:

$$x ( n ) = 2 * \frac{x ( n ) - max ( x ( n ) )}{max ( x ( n ) ) - min ( x ( n ) )} - 1$$

When needed, this normalization technique can be used to provide signal processing techniques with a suitable range of values. Also, the [−1,+1] normalization can be used for visualization purposes when there is the need to display more than one measure of variability. It may be necessary to bind the range of values to effectively display their dynamic into a monitor. The simultaneous representation of multiple measures could be useful for a qualitative assessment of the variability trends (each measure of variability is likely to behave in different ways over time).

When a wide array of variability measures is employed, it is possible to reduce their number through specific criteria applied during the feature selection stage 106. The criteria that may be used to select a subset of the variability parameters may include, without limitation:

1. Domain-specific selection 118: one or more of the available domains of variability are selected, neglecting the other domains. Possible domains are the scale-invariant, the frequency, the statistical, etc. This selection enables the investigation of the value of the individual domains of variability in clinical applications.

2. Physiologically-based selection 120: only the measures with a given physiological explanation are selected. An example is the selection of variability measures based on a frequential representation of the original physiological time series; those measures are indeed known to be related with brain activity. Therefore, this selection enables the association of physiological phenomena to clinical events.

3. Clinically relevant selection 122: only the measures that were found to be associated with a specific clinical outcome are selected. For instance, it has been found that certain variability measures continuously drop down beginning 36 hours before the development of septic shock. Therefore, the selection of those measures would lead to a composite variability 20 specific to the tracking of sepsis. Alternatively, a correlation-based selection between each single measure and the expected trend in variability could be used. Only the measures with a correlation higher than a certain threshold would be selected.

It can therefore be appreciated that different types of normalization and feature selection approaches can be implemented, depending on the information of interest.

In order to incorporate multiple variability measures 14 into a composite variability 20, the integration stage 108 is performed. The integration stage 108 is used to fuse the variability measures 14 that survived the selection process 106 (if applied).

There are various possible techniques of integration, for example, the PCA technique 126, the ICA technique 128, and the median 130 of the variability. PCA 126 is a signal processing technique which captures the direction of maximum variance within a dataset, as well as the directions uncorrelated to the dataset. Therefore, PCA 126 allows the creation of a composite measure of variability 20 assessing the degree of change in variability. ICA 128 on the other hand can be used to find a set of independent signals generating the recorded variability. Therefore, ICA 128 is capable of identifying the underlying stimuli causing variability (e.g. circadian fluctuations, sympathetic/parasympathetic activity). The median 130, computed across the values of the measures of variability at a given time, gives a conservative measure of the entity of variation.

It may be noted that the type of integration used in stage 108, for example, PCA 126, ICA 128, or median 130, will depend on the intent of the application. For example, when the application intends on characterizing changes in variability that occurs as a result of a clear known time stamped intervention (e.g. spontaneous breath trial (SBT) or sedation vacation) the ICA technique 128 is likely to be preferred, since the variability parameters that change at the time of the event can be selected, and a composite of those measures built. When there is ongoing monitoring and the application is attempting to detect something without an a priori known time point (e.g. an onset of infection), then the PCA technique 126 or the median technique 130 may be preferred. When one is trying to pick up a sensitive measure of a change in any variability measures 14, then PCA 126 is likely preferred. When one wants to detect a change in a large number of variability measures 14, then median 130 is likely preferred. Accordingly, the principles discussed herein provide a flexible, tunable, selective approach with various integration techniques for multiple clinical scenarios.

These integration methods, used in the present context, create a mathematical model of variability. It may be noted that the mathematical model can be based on other patient information (i.e. over and above the physiological variability determined from the patient), For example, various “clinical parameters” (e.g., age, gender, level of glucose in the blood, etc.) may be considered.

It can be appreciate that the integration stage 108 allows the convergence of the information of a given subject, into a unique measure (i.e. the composite). The mathematical model generated by the integration is subject-specific, making the composite measure of variability 20 “individualized”. It is however possible to combine multiple individualized models to generate a population model, wherein each individualized model would come from a different patient). Such a population model can be used to create a composite measure of variability 20, from which is possible to create a decision model, which gives a probability of detecting/predicting a certain clinical outcome. When creating the decision model, it is possible to use clinical parameters (i.e. another type of patient information), to improve the detection/prediction capabilities of the system.

Accordingly, an individualized model can be used, as well as a population model. The individualized model should be useful to monitor the conditions of a patient in situations where there is no population model of the changes in variability for that specific clinical situation (e.g., when monitoring septic patients, when in the literature no one has published anything about changes in variability due to sepsis). The selection of the type of individualized model (e.g. selecting PCA rather than ICA), is based on a clinical assumption, e.g. it is expected to see a drift from the baseline of health when a subject develops sepsis. Instead, the population model is based on multiple individualized models, and provides additional information such as detection/prediction, at the expense of being “not individualized” anymore. To provide an example: assume the heart rate variability of “John” is being monitored while he is developing sepsis. The composite measure of variability 20 generated through an individualized model may show a quick drop at 10:05 am, however the composite measure of variability 20 generated through the population model may drop only at 10:20 am. This may be likely to happen because the population model will be more sensitive to the change of the “average person” (the average of the population), and less sensitive to the change of the “far from average person” (the tails of the population distribution). However, from the population-based composite measure of variability 20, it is possible to generate a decision model, which gives the probability that John is developing sepsis while monitored.

It can also be appreciated that it is possible to obtain the probability that a patient is changing is clinical conditions (e.g. deterioration) by using only the individualized model (by applying to the composite a so called “single-class classifier”). Moreover, the population model may be created by looking at the variability of all the subjects at the same time, rather than combining a set of individualized models.

It can also be appreciated that the mathematical model will vary based on the number of variability measures 14 used, the application, and the integration techniques used. For example, integration techniques such as PCA 126 and ICA 128 techniques may apply a linear combination of the variability measures 14, i.e. multiplying each measure by a specific coefficient, and then summing the measures to obtain a unique time series.

From the integration stage 108, it is possible to create one or more composite measures of variability 20. For example, PCA 126 and ICA 128 techniques create multiple principal and independent components. It is therefore possible to select only one of those components using the component selection sub-stage 134, and the selected components. Alternatively, it is possible to select one or more of those, and estimate the probability of development of a specific clinical outcome (also referred to herein as clinical likelihood—discussed below).

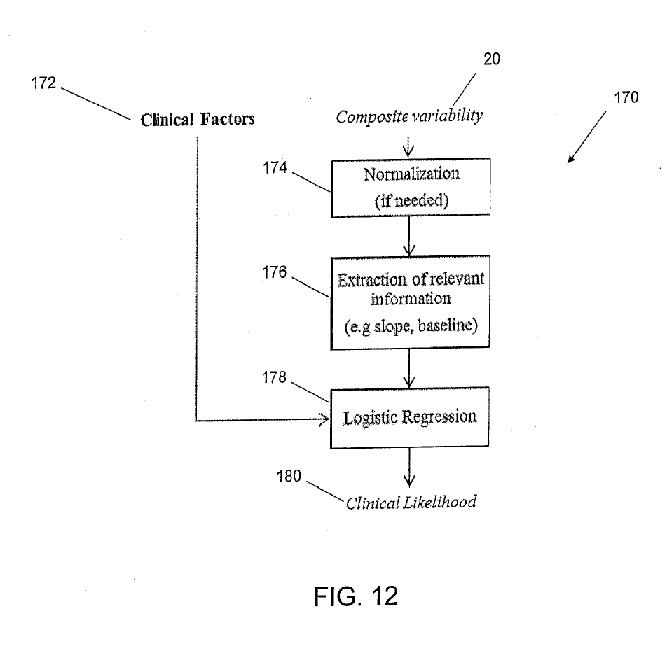

An example procedure 170 to estimate the clinical likelihood 180 using a composite variability 20 is shown in FIG. 12. The procedure 170 shown in FIG. 12 can be applied to any clinical outcome detectable through variability analysis, and includes the following steps:

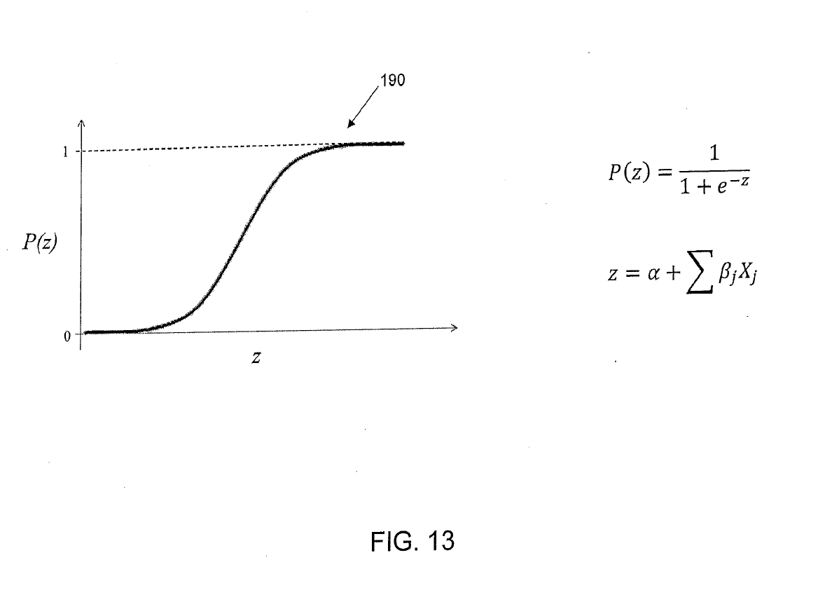

At step 174, the composite variability time series is/are normalized, if necessary or desired in the particular application. At step 176, relevant features are extracted from the normalized time series (e.g. slope of the curves, sample mean, sample standard deviation, etc.). At step 178, a technique taken from the domain of Pattern Analysis is used to create the clinical likelihood 180, by using the composite variability, and joining to that information the one coming from several clinical factors 172 (e.g., body temperature, Body Mass Index, etc.). As illustrated in FIG. 12, Logistic Regression is a Pattern Analysis technique suitable for this purpose. The Logistic Regression technique creates, through a linear model, a variable, which is in FIG. 13. The variable z is therefore transformed through a logistic function P(z), into a probability. For example, the following formula, also shown in FIG. 13 may be used:

$$P ( z ) = \frac{1}{1 + {e}^{- z}} ,$$

where: z=α+ΣβⱼXⱼ. In FIG. 13 it may be noted that α and β are constants, while x is a vector made joining the composite variability features with the clinical factors. It may also be noted that Logistic regression is a deeply characterized technique widely used in clinical applications, providing statistical information on the estimated probability (e.g. confidence intervals, predictive intervals).

In the following, examples of applications of for a composite variability 20 are provided. The applications of the composite variability 20, and the clinical likelihood 180, include the scenarios discussed below, as well as various others. As such, the following examples are for illustrative purposes only. In each scenario presented below, it is assumed that the variability measures 14 are extracted from multiple physiological signals, from at least two organ systems.

A. Tracking of the Development of Sepsis

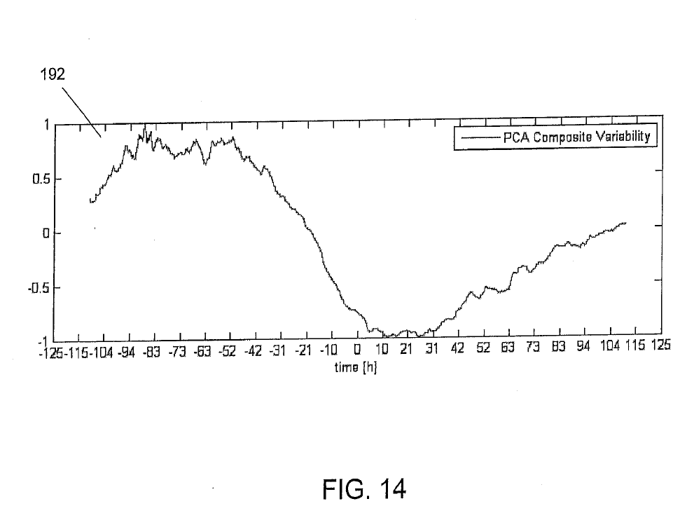

Consider a set of variability measures recorded from a patient that just underwent a bone marrow transplant. It has been shown, in the case of sepsis development, that the patient variability continuously drops down in the days following intervention. Using an admission condition normalization 110, without any particular selection, and integrating the information through a PCA technique 126, it may be possible to track the development of sepsis as shown in FIG. 14. It may be noted that FIG. 14 illustrates the tracking of sepsis centered at the time of sepsis detection (t=0).

It may be noted that the admission condition normalization 110 can enhance the change from a healthy baseline, and the PCA technique 126 can detect the direction of maximum variation of variability. This composite variability 20 would be specific for the detection of the occurrence of sepsis, promoting a faster and more effective care.

B. Estimation of the Probability of Extubation Outcome

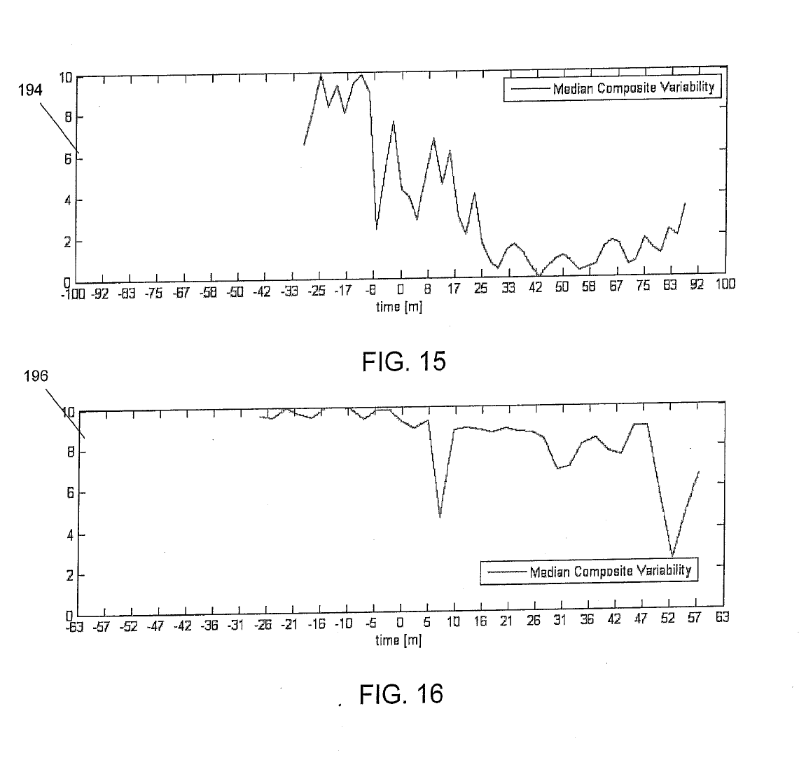

Consider a set of variability measures recorded from a patient that is under mechanical ventilation, and that will undergo a SBT—a procedure used to assess whether the patient can be successfully extubated). It has been found that there is a drop of variability during the SBT, and this quantity can be used to assess the probability of extubation outcome. Using the population-based normalization 112, selecting a set of measures known to be related with this clinical outcome (i.e. clinically relevant selection), and integrating the measures of variability with the median, a composite measure describing the degree of change during a SBT would be created as shown in FIGS. 15 and 16—graphs 194 and 196. It may be noted that FIGS. 15 and 16 illustrate trends of the composite variability 20 for two patients monitored before and during an SBT trial. The graph 194 in FIG. 15 corresponds to a failed extubation and the graph 196 in FIG. 16 shows a passed extubation. At time 0, the SBT started. The composite variability 20 in this example was created using the population-based normalization 112, a clinically-relevant selection 122 based on previous research, and a median integration 130.

The combination of the composite measure of variability with clinical factors (e.g. tidal volume, rapid shallow breathing index) in a logistic regression model would lead to a likelihood of successful extubation. This likelihood could be used by the physicians during the SBT to decide whether it is more valuable to keep the patient in the SBT phase, or to stop the SBT and either extubate or keep the patient intubated. Therefore, the patient's care would be improved and care costs would be diminished.

In FIGS. 17 to 19, examples of population trends of three measures of variability are shown. In FIG. 17, a first graph 200 of a Shannon Entropy (shannEn) trend is shown, in FIG. 18, a second graph 202 of a Plotkin-Swamy energy operator (PSeo) is shown, and in FIG. 19, a third graph 204 of a Fuzzy entropy (fuzEn) trend is shown. These measures were applied to eight populations: subjects healthy and young (HY), healthy and old (HO), unhealthy and young (UY), unhealthy old (UO), patients that passed extubation before a spontaneous breath trial (pSBT), patients that failed extubation during a spontaneous breath trial (dSBT), patients six hours before the diagnosis of sepsis (6bS), patients six hours after the diagnosis of sepsis (6aS). As showed, there is a monotone relationship between the degree of impairment and the value of variability.

C. Sympathetic/Parasympathetic Activity Estimation

The sympathetic and parasympathetic activities of the brain regulate the heartbeat. Therefore, given the appropriate tools, it may be possible to indirectly measure these brain activities from heart rate variability (HRV). It has been found that through HRV it is possible to assess the degree of severity of Parkinson's disease. A composite measure of variability 20 useful for this purpose could make use of the [+1,−1] normalization 114 (needed to let the measures be comparable with each other), a selection of the measures of variability directly estimating the brain activity (i.e. physiologically-based selection 120) and the ICA integration technique 128, which would identify and select either the sympathetic or the parasympathetic component affecting variability. Some variability measures associated with parasympathetic activity are the power of an RR interval time series at high-frequency (0.15-0.40 Hz) and the SD2 index extracted from Poincaré plots; some associated to both sympathetic and parasympathetic activities are the low-frequency/high-frequency ratio, the SD1 index from the Poincaré plots and the Number of Variations from Symbolic dynamics.

This composite variability 20 could open the way to the creation of a set of clinically-oriented applications of variability for brain diseases, as well as represent a valuable research tool to understand the physiology of the brain. Some studies using ICA for this purpose already exist, however they directly applied this method to RR interval time series, and not the variability measures extracted from it.

The principles described herein provide a procedure to create a variety of clinically relevant time series, namely composite measures. Each composite measure describes in a continuous and individualized way the conditions of patient. Each composite measure is based on the continuous analysis of a variety of variability time series extracted from a variety of physiological signals. Each composite measure provides independent and clinically valuable information. Therefore, the methods described herein provide several measures that can be used alone or together, depending on the specific clinical outcome of interest, so as depending on the clinical needs of the patient.

Moreover, the principles discussed herein represent a further step along the path from a descriptive to a predictive medicine. The assessment at an early stage of the continuous deterioration of a patient's physiological condition would allow the prevention of further impairment. Better probabilistic prediction of the time at which a patient can be removed from e.g. a life support system can significantly decrease the costs of clinical care associated with certain critical conditions.

It will be appreciated that any module or component exemplified herein that executes instructions may include or otherwise have access to computer readable media such as storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by an application, module, or both. Any such computer storage media may be part of the composite variability component 10, variability analysis component 12, etc., or accessible or connectable thereto. Any application or module herein described may be implemented using computer readable/executable instructions that may be stored or otherwise held by such computer readable media.

It will also be appreciated that the example embodiments and corresponding diagrams used herein are for illustrative purposes only. Different configurations and terminology can be used without departing from the principles expressed herein. For instance, components and modules can be added, deleted, modified, or arranged with differing connections without departing from these principles.

The steps or operations in the flow charts and diagrams described herein are just for example. There may be many variations to these steps or operations without departing from the spirit of the invention or inventions. For instance, the steps may be performed in a differing order, or steps may be added, deleted, or modified.

Although the invention has been described with reference to certain specific embodiments, various modifications thereof will be apparent to those skilled in the art without departing from the spirit and scope of the invention as outlined in the claims appended hereto.

1. A method comprising:
obtaining a plurality of measures of variability; and
combining the plurality of measures of variability to obtain a composite measure of variability.

2. The method of claim 1, wherein at least one of the plurality of measures of variability corresponds to a single organ.

3. The method of claim 1, wherein at least one of the plurality of measures of variability corresponds to multiple organs.

4. The method of claim 1, further comprising tracking the composite measure of variability over time.

5. The method of claim 1, wherein the composite measure of variability reflects an overall degree of change in at least one physiological condition.

6. The method of claim 1, further comprising correlating the composite measure of variability with at least one clinical outcome.

7. The method of claim 1, wherein the composite measure of variability is sensitive to physiological and pathological stimuli causing variability.

8. The method of claim 1, further comprising obtaining a plurality composite measures of variability and combining the plurality composite measures of variability with clinical information to produce a likelihood of development of at least one clinical outcome.

9. The method of claim 1, further comprising tuning the composite measure of variability to detect at least one clinical outcome by enabling selection of any one or more of normalization, selection, and integration procedures applied to the plurality of variability measures.

10. The method of claim 1, further comprising tuning the composite measure of variability to investigate at least one physiological phenomenon underlying variability by enabling selection of any one or more of normalization, selection, and integration procedures applied to the plurality of variability measures.

11. The method of claim 1, the combining the plurality of measures of variability to obtain a composite measure of variability comprising:
normalizing the plurality of variability measures; and
applying an integration procedure to the plurality of variability measures to generate the composite measure of variability.

12. The method of claim 11, the combining the plurality of measures of variability to obtain a composite measure of variability further comprising filtering the plurality of measures of variability prior to the normalizing.

13. The method of claim 11, the combining the plurality of measures of variability to obtain a composite measure of variability further comprising selecting a subset of variability parameters subsequent to the normalizing.

14. A computer readable medium comprising computer executable instructions for:
obtaining a plurality of measures of variability; and
combining the plurality of measures of variability to obtain a composite measure of variability.

15. A system comprising a processor coupled to a memory, the memory comprising computer executable instructions for:
obtaining a plurality of measures of variability; and
combining the plurality of measures of variability to obtain a composite measure of variability.
